(19) United States
(12) Patent Application Publication
(10) Pub. No.: US 2003/0176907 A1
(43) Pub. Date: Sep. 18, 2003

(54) Ventricular pacing lead device

(76) Inventor: Mark Tarvin, Columbus, GA (US)

Correspondence Address: MYERS & KAPLAN, INTELLECTUAL, PROPERTY LAW, L.L.C., 1827 POWERS FERRY ROAD, BUILDING 3, SUITE 200,, ATLANTA, GA, 30339, US

(21) Appl. No.: 10/100,415

(22) Filed: Mar. 18, 2002

(51) Int. Cl. A61N 1/05

(52) U.S. Cl. 607/126

(57) ABSTRACT

A ventricular stylet and pacing lead having a novel, modified curve shape enabling easy placement and positioning, a uniquely oversized torque handle enabling efficient manipulation and an internally carried, arcuately barbed tip enabling minimally traumatic fixation within the heart, thereby decreasing total implant time, decreasing ventricular lead placement time and facilitating better apical placement in the right ventricle, thus lowering thresholds.

### TECHNICAL FIELD

The present invention relates generally to medical lead devices and, more specifically, to a ventricular stylet and pacing lead having a curved shape enabling easy placement and positioning, a torque handle enabling efficient manipulation and an internally carried, ejectable semi-helical barbed tip enabling minimally traumatic fixation within the heart.

### BACKGROUND OF THE INVENTION

Pacer placement and utilization requires internal placement of an electrode with connection to a pacing unit. A first end of the electrode is connected to a pacing unit and a second end of the electrode is conductively related to the heart wall. Typical electrodes are elongated conductive wires surrounded by a flexible sheath, catheter or stylet.

Curvilinear atrial electrodes are known, wherein effective placement within the atrium requires complex manipulation through the superior vena cava. Curvature in ventricular electrodes, however, has heretofore been deemed unwarranted because insertion of a linear lead into the ventricle was reported as “simple” and “without difficulty”. Moreover, utilization of presently available “J” curve atrial leads as ventricular leads can be traumatic, and is disadvantageous in view of the novel ventricular curvature of the present invention.

Effective placement of a pacing electrode also depends upon proper manipulation, wherein a physician can be required to reorient the electrode tip within the heart prior to final placement. One common lead design requires a physician to utilize both hands in order to accomplish any manipulation, one hand to hold stylet tubing and the other hand to rotate the lead. Occasionally, small stylet handles or generally extended lead tips are provided to assist with gripping, however, these are disadvantageous in view of the present invention.

Once the pacing electrode is successfully positioned at the targeted location, means of retaining the position is typically provided via active fixation to the heart wall. Screw-type tips are known, wherein aggressive engagement with the heart wall is accomplished by multiple revolutions of the fixative end.

Unfortunately, this design can lead to perforation of the heart wall. Pre-wound tips have been suggested in an attempt to avoid such damage. However, placement of such tips still disadvantageously requires aggressive screwing into the heart wall.

One presently available alternative to the screw-type fixative ends is the utilization of fixed tines. Such tines are intended to cooperate with the trabeculae of the right atrial appendage in lieu of screwing in to the heart wall, but, like the curved lead shape, have not been deemed necessary for ventricular placement. Moreover, fixed position tines that continuously extend from the tip of the electrode are disadvantageous because premature engagement may occur during placement. Extendable non-conductive tines have been suggested, wherein a protective shroud holds the tines in a non-extended state until retraction of the shroud enables full extension. This design is also disadvantageous in view of the present invention, however, because the tines are held in position on the exterior of the surface of the lead and as such, remain partially exposed to potential contamination prior to and during the placement procedure. Likewise, the retraction mechanism for the protective shroud also resides on the external surface of the lead stylet. Thus, in order to release the tines, the shroud must move along the exterior surface, potentially contacting and/or damaging surrounding tissues and possibly increasing risk of infection.

Therefore, it is readily apparent that there is a need for a ventricular stylet and pacing lead having a curved shape enabling easy placement and positioning, an oversized torque handle enabling efficient manipulation and an internally carried, semi-helical barbed tip enabling minimally traumatic fixation within the heart, thus preventing the above-discussed disadvantages.

### BRIEF SUMMARY OF THE INVENTION

Briefly described, in a preferred embodiment, the present invention overcomes the above-mentioned disadvantages and meets the recognized need for such a device by providing a ventricular curve stylet wire with an oversized torque handle and a modified, preformed semi-circular curve shaped ventricular pacing lead having deployable, semi-helical active fixation tines, wherein exceptional facilitation through small superior vena cava is achieved, superb manipulation across the tricuspid valve is realized and atrial wall migration and perforation is greatly reduced.

According to its major aspects and broadly stated, the present invention is a ventricular stylet and pacing lead having a novel, modified curve shape enabling easy placement and positioning, a uniquely oversized torque handle enabling efficient manipulation and an internally carried, arcuately barbed tip enabling minimally traumatic fixation within the heart, thereby decreasing total implant time, decreasing ventricular lead placement time and facilitating better apical placement in the right ventricle, thus lowering thresholds.

More specifically, the present invention is a pre-shaped ventricular curve stylet wire and pacing lead, wherein the device is generally semi-circularly shaped in a unique modification of an atrial “J” curve. The extended curvature of the generally semi-circularly shaped wire offers superb manipulation across the tricuspid valve into the right ventricle and provides for less traumatic placement than would otherwise be realized through the utilization of an atrial “J” lead. The curvature of the wire also enables easy cannulation of the coronary sinus for biventricular pacing. Moreover, the preferred stiffness of wire results in an amount of curve loss ideal for crossing the tricuspid valve into the right ventricle. The pre-shaped ventricular curve stylet wire and pacing lead are preferably formed with several variations in overall length, wire diameter and curve size to enable effective and comfortable placement and utilization in a variety of body sizes.

A large, user-friendly torque handle is provided at the distal end of the stylet wire, wherein the torque handle with its grip-enhancing surface enables superb manipulation through virtually any small superior vena cava or venous channel. A unique deployment and locking mechanism is provided at the distal end of the ventricular curve pacing lead, wherein atraumatic, semi-helical tines affix into the heart wall and act as a cushion to prevent further migration, decreasing the chance of perforation. This arcuately tined active fixation mechanism, coupled with the anatomically correct preformed ventricular curve encourages better values at implant.

A feature and advantage of the present invention is the ability of such a device to enable a heretofore unrealized combination of benefits including, but not limited to, decreased total implant time, decreased ventricular lead placement time and better apical placement in the right ventricle, thereby lowering thresholds.

Another feature and advantage of the present invention is the ability of such a device to enable easy and efficient ventricular placement, positioning and manipulation.

Another feature and advantage of the present invention is the ability of such a device to enable minimally traumatic fixation within the heart.

Another feature and advantage of the present invention is the ability of such a device to reduce atrial wall migration and perforation.

Another feature and advantage of the present invention is the ability of such a device to enable exceptional facilitation through small superior vena cava.

Another feature and advantage of the present invention is the ability of such a device to enable superb manipulation across the tricuspid valve.

Another feature and advantage of the present invention is the ability of such a device to provide for less traumatic placement than would otherwise be realized through the utilization of an atrial “J” lead.

Another feature and advantage of the present invention is the ability of such a device to enable easy cannulation of the coronary sinus for biventricular pacing.

Another feature and advantage of the present invention is the ability of such a device to enable an amount of curve loss ideal for crossing the tricuspid valve into the right ventricle.

Another feature and advantage of the present invention is the ability of such a device to be effectively and comfortably placed and utilized in a variety of body sizes.

Another feature and advantage of the present invention is the ability of such a device to enable enhanced gripping.

Another feature and advantage of the present invention is the ability of such a device to encourage better values at implant

These and other objects, features and advantages of the invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a top view of a ventricular pacing lead apparatus according to a preferred embodiment of the present invention.

FIG. 2a is a partial cutaway, cross-sectional view of a ventricular stylet and pacing lead according to a preferred embodiment of the present invention, showing a non-deployed fixation tip.

FIG. 2b is partial cutaway view of the ventricular stylet and pacing lead of FIG. 2a, showing a deployed fixation tip.

FIG. 3a is an end view of a ventricular stylet and pacing lead apparatus according to a preferred embodiment of the present invention, showing a non-deployed fixation tip.

FIG. 3b is a side view of a ventricular pacing lead according to a preferred embodiment of the present invention, showing a deployed fixation tip.

FIG. 3c is a partial cutaway, cross-sectional view of a ventricular stylet and pacing lead according to a preferred embodiment of the present invention, showing a fixation tip positioned on a heart wall.

FIG. 4 is a partial cross-sectional view of a ventricular stylet and pacing lead according to a preferred embodiment of the present invention, showing a deployed fixation tip.

FIG. 5 is a top view of a ventricular pacing lead apparatus according to a preferred embodiment of the present invention, showing preferred dimensional characteristics and reference points.

### DETAILED DESCRIPTION OF THE PREFERRED AND ALTERNATE EMBODIMENTS

In describing the preferred and alternate embodiments of the present invention, as illustrated in the figures and/or described herein, specific terminology is employed for the sake of clarity. The invention, however, is not intended to be limited to the specific terminology so selected, and it is to be understood that each specific element includes all technical equivalents that operate in a similar manner to accomplish similar functions.

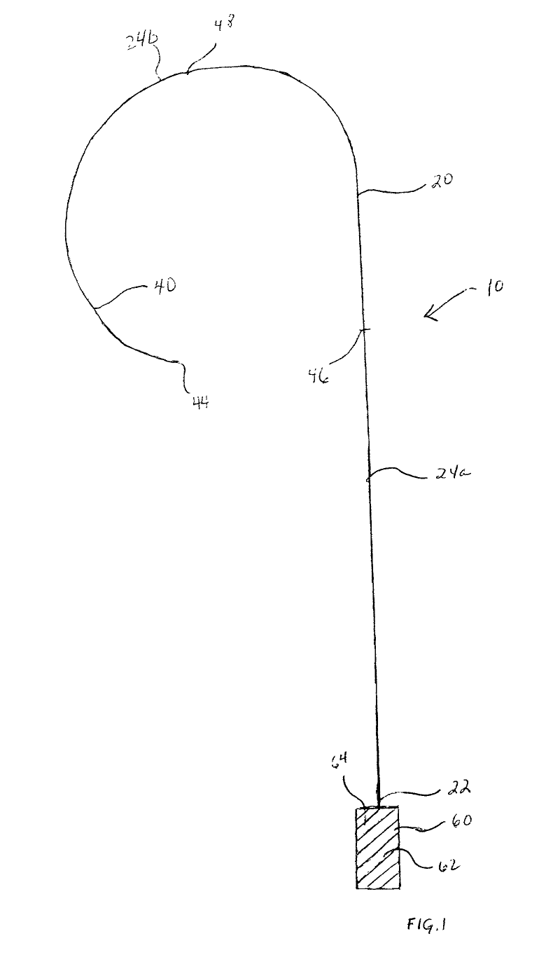

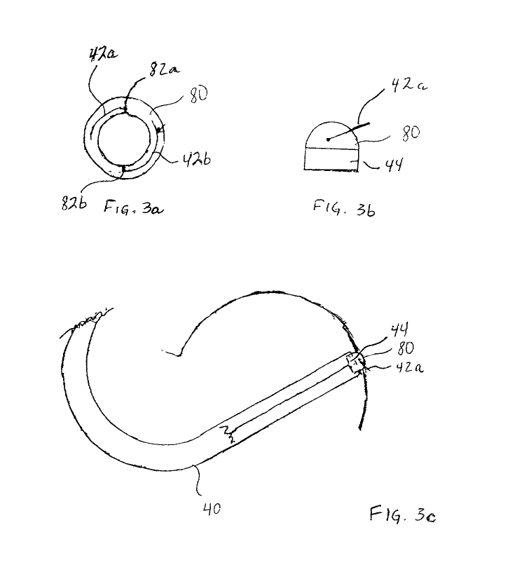

Referring now to FIG. 1, the present invention is ventricular pacing lead device 10 having curved lead guide wire or stylet 20, pacing lead 40 and torque handle 60, wherein torque handle 60 is positioned on proximate end 22a of stylet 20. As best seen in FIGS. 3a and 3b, pacing lead 40 has at least one generally arcuately shaped tine 42a positioned proximate to distal end 44. Preferably, two generally arcuately shaped tines 42a and 42b are opposingly positioned proximate to distal end 44 of pacing lead 40. However, one skilled in the art would readily recognize that, although two tines 42a and 42b are preferred, more than two tines could be utilized without departing from the intended scope and function of the present invention.

Referring now to FIG. 1, curved lead guide wire or stylet 20 is preferably defined by first generally linear segment 24a and second generally semi-circular segment 24b, wherein the shape thereof is preferably pre-formed. Second generally semi-circular segment 24b is a novel and unique modification of a known atrial “J” curve, wherein extended curvature is realized proximate to distal end 44 of pacing lead 40. Preferably, the resiliency of guide wire or stylet 20 provides flexibility of curvature to enable curve loss during passage and curve reformation during placement.

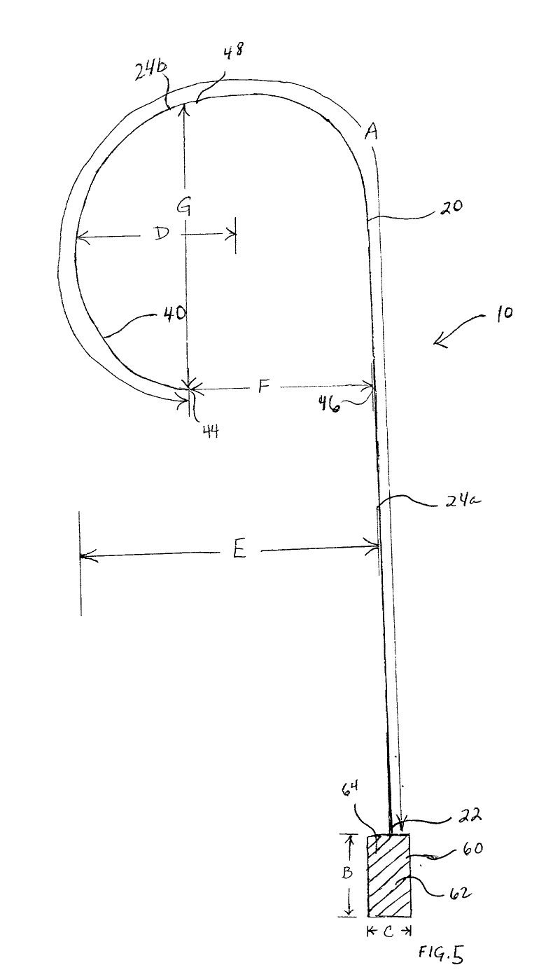

The pre-shaped ventricular curve stylet wire 20 is preferably formed of stainless steel, platinum or titanium, with several variations in overall length, wire diameter and curve size to enable effective and comfortable placement and utilization in a variety of body sizes. Three sizes are preferred and are necessary to enable the realization of the unique combination of benefits presented by the invention herein described, however, other sizes may also be utilized to accommodate smaller or larger patients or to assist with other user preferred manipulation techniques. The three preferred sizes include small, medium and large versions, wherein dimensional specifications define preferred embodiments for each size, as best seen in FIG. 5.

A preferred small size embodiment may be formed from either firm stainless steel wire having a diameter of approximately 0.014 inches to 0.015 inches or soft stainless steel wire having a diameter of approximately 0.012 inches to 0.013 inches. The preferred total length A of wire 20 is approximately 52 centimeters, wherein torque handle 60 is preferably approximately 2 centimeters in length B and 1 centimeter in width C. Preferably, semi-circular segment 24b of the small size embodiment has radius D of approximately 3 centimeters and diameter E of approximately 6.1 centimeters, wherein the preferred open distance F between distal end 44 and base 46 of semi-circular segment 24b is approximately 3.5 centimeters and the preferred curvature defines an arc having approximately 6 centimeter base G extending between distal end 44 and point 48 on semi-circular segment 24b.

A preferred medium size embodiment may be formed from either firm stainless steel wire having a diameter of approximately 0.014 inches to 0.015 inches or soft stainless steel wire having a diameter of approximately 0.012 inches to 0.013 inches. The preferred total length A of wire 20 is approximately 58 centimeters, wherein torque handle 60 is preferably approximately 2 centimeters in length B and 1 centimeter in width C. Preferably, semi-circular segment 24b of the medium size embodiment has radius D of approximately 3.5 centimeters and diameter E of approximately 7.3 centimeters, wherein the preferred open distance F between distal end 44 and base 46 of semi-circular segment 24b is approximately 5.5 centimeters and the preferred curvature defines an arc H having an approximately 7 centimeter base G extending between distal end 44 and point 48 on semi-circular segment 24b.

A preferred large size embodiment may be formed from either firm stainless steel wire having a diameter of approximately 0.014 inches to 0.015 inches or extra firm stainless steel wire having a diameter of approximately 0.015 inches to 0.017 inches. This large size embodiment offers unexpectedly exceptional results for crossing the tricuspid valve in larger body frames and has proven to be excellent in patients with congestive heart failure in which the heart is enlarged. The preferred total length A of wire 20 is approximately 60 centimeters, wherein torque handle 60 is preferably approximately 2 centimeters in length B and 1 centimeter in width C. Preferably, semi-circular segment 24b of the large size embodiment has radius D of approximately 4.4 centimeters and diameter E of approximately 8.9 centimeters, wherein the preferred distance between distal end 44 and base 46 of semi-circular segment 24b is approximately 6.8 centimeters and the preferred curvature defines arc H having an approximately 8.1 centimeter base G extending between distal end 44 and point 48 on semi-circular segment 24b.

It should be readily recognizable to one skilled in the art that, while the dimensional specifications are preferred for three size embodiments of the present invention, other dimensions may also be utilized without departing from the intended scope of the present invention, wherein the novel, overall semi-circular shape remains for ventricular pacing lead device 10.

As previously described, torque handle 60 is preferably 2 centimeters in length B and 1 centimeter in width C, preferably with a generally cylindrical shape. It should be noted that a key feature of torque handle 60 is the large size, wherein variations to the overall shape would not depart from the anticipated scope of the present invention and variations in size could also be incorporated while maintaining the general oversized nature. Preferably, outer surface 62 of torque handle 60 has grip-enhancing pattern 64 provided thereon, wherein grip-enhancing pattern 64 has diagonal striations, preferably positioned at approximately 45°. Although a 45° grip-enhancing pattern is preferred, other types and designs of grip-enhancements could also be utilized such as, for exemplary purposes only, a stipple pattern, a cross-hatch pattern, a plurality of parallel lines, a rubberized coating or any other grip-enhancing means or surface.

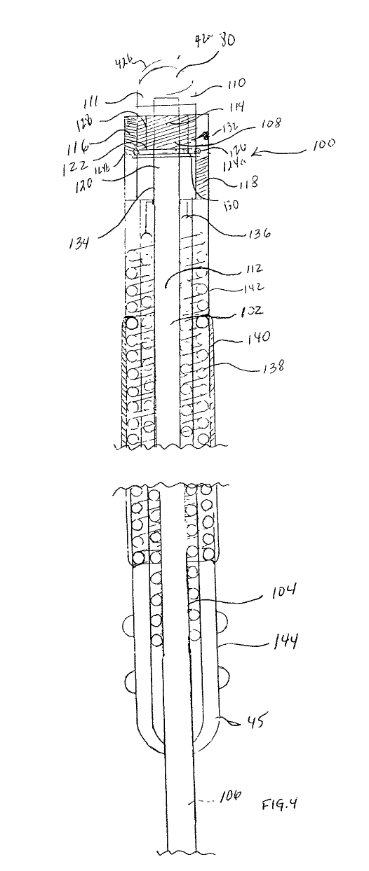

As best seen in FIGS. 3a, 3b and 4, ventricular pacing lead device 10 preferably has a unique substantially internally positioned deployment and locking mechanism 100 for fixation tip 80, wherein fixation tip 80 is positioned proximate to distal end 44 of pacing lead 40. Fixation tip 80 preferably has a steroid coating, however, can alternately be provided without a steroid coating. Preferably generally arcuately shaped tines 42a and 42b are positioned on fixation tip 80, wherein first attachment ends 82a and 82b of tines 42a and 42b, respectively, are preferably opposingly positioned. Such placement of tines 42a and 42b, along with the arcuate shape thereof, enables formation of a semi-circular or semi-helical pattern. Preferably, tines 42a and 42b are formed from conductive metal.

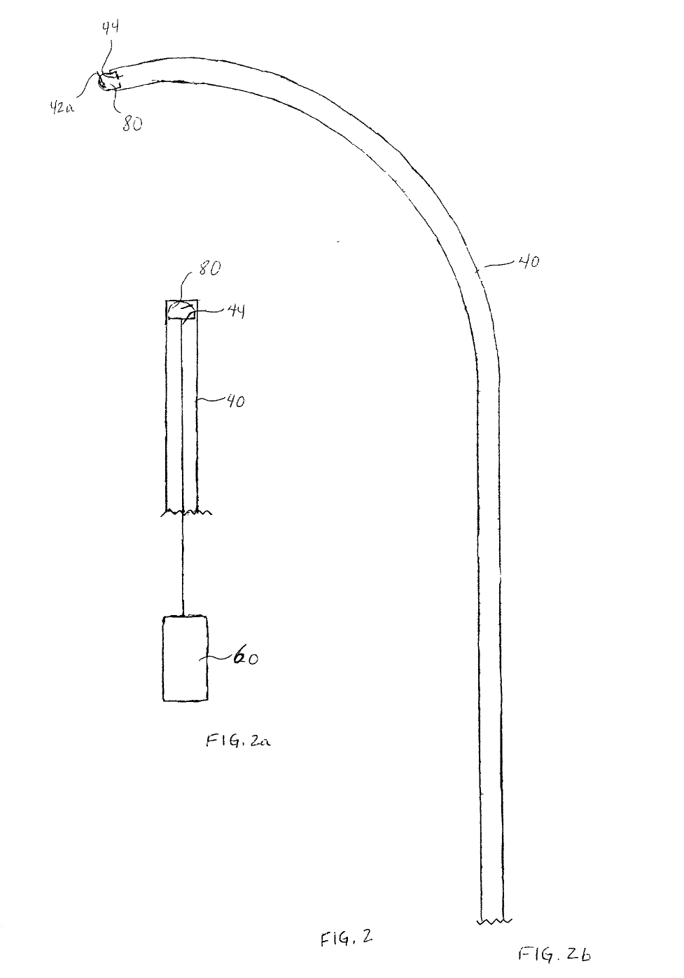

Prior to deployment, fixation tip 80 along with tines 42a and 42b is preferably positioned or retracted substantially within distal end 44 of pacing lead 40, as best seen in FIG. 2a. After deployment, fixation tip 80 along with tines 42a and 42b is preferably positioned substantially external to distal end 44 of pacing lead 40, as best seen in FIG. 2b. Moreover, after deployment, tines 42a and 42b preferably and generally cease to be arcuately shaped. Upon exit from distal end 44 of pacing lead 40, each tine 42a and 42b extends outwardly in a generally linear fashion, as best seen in FIG. 3b.

As best seen in FIG. 3c, deployment of fixation tip 80 enables atraumatic semi-helical tines 42a and 42b to affix into the heart wall or to the trabeculae of the atrial appendage and to act as a cushion to prevent further migration, decreasing the chance of perforation.

Deployment and locking mechanism 100, best seen in FIG. 4, enables user control of presentation of tines 42a and 42b to the heart, wherein retracted fixation tip 80 and tines 42a and 42b reside within distal end 44 of pacing lead 40. Preferably hollow stylet core 102 extends longitudinally within pacing lead 40, wherein proximate to proximal end 106, preferably stainless steel connector 104 is recessed therewithin. Core surface 112 preferably has a silicone, NINTONOL and/or TEFLON coating to facilitate movement of stylet wire 20 therewithin. Proximate to distal end 108 of pacing lead 40, stylet core 102 generally extends within preferred cone body 110 to core surface 114, wherein core surface 114 within cone body 110 is preferably metal and preferably defines a substantially square shape 120 proximate to base 122 of cone body 110, and wherein cone body 110 is preferably electrically conductive and composed of platinum, platinum/iridium, titanium, titanium/carbon, stainless steel or any other appropriately conductive metal, metals or alloys.

First retraction spring 116 is preferably positioned proximate to distal end 44 of pacing lead 40, proximate to outer surface 111 of cone body 110 and threaded member 128, wherein second o-ring locking device 124b is positioned proximate thereto. Second deployment spring 118 is preferably positioned proximate to distal end 44 of pacing lead 40, proximate to outer surface 111 of cone body 110 and threaded member 128, wherein first o-ring locking device 124a is positioned proximate thereto. Preferably silicone o-ring 126 is positioned proximate to base 130 of threaded member 128, wherein deployment and locking mechanism 100 urges compression of second deployment spring 118 and silicone o-ring 26 is held in a generally fixed position between first o-ring locking device 124a and second o-ring locking device 124b. Preferably, thread receiver 132 performs as a deployment mechanism screw cam, generally interacting with threaded member 128 to deploy fixation tip 80 and tines 42a and 42b.

Preferably, electrical conducting strip 134 generally surrounds the periphery of square shape 120 of stylet core 102 and connects to inner wire coil conductor 136, wherein inner wire coil conductor 136 is preferably positioned proximate to stylet core 102. The preferred polyurethane, silicone insulating coating of inner wire coil conductor 136 prevents undesirable electrical cross-talk between inner wire coil conductor 136 and outer coil 138, wherein outer coil 138 is preferably wrapped around inner coated coil 136.

Preferably, bipolar electrical band configuration 140 is positioned on outer surface 142 of pacing lead 40, proximate to outer coil 138, wherein conductive outer coil 138 is a bipolar component. The preferred composition of bipolar electrical band configuration 140 is metal such as, for exemplary purposes only, stainless steel, platinum, platinum/iridium, titanium or any other suitable metal or alloy.

Preferably, suture anchor sleeve 144 is provided on outer surface 142 of pacing lead 40 proximate to proximal end 106 of pacing lead 40, wherein silicone is provided to prevent body fluid migration. Proximal end 106 of pacing lead 40 is preferably dimensioned to connect the bipolar component to the pacing generator.

In an alternate embodiment, pacing lead 40 could have only one generally arcuately shaped tine 42a positioned proximate to distal end 44.

In an alternate embodiment, pacing lead 40 could have more than two generally arcuately shaped tines 42a and 42b.

In an alternate embodiment, a small size embodiment may be formed from stainless steel wire of any firmness and/or diameter.

In an alternate embodiment, a small size embodiment may be formed from any length of wire.

In an alternate embodiment, a small size embodiment may be formed with a radius D of less than or greater than 3 centimeters and a diameter E of less than or greater than 6.1 centimeters.

In an alternate embodiment, a small size embodiment may be formed with an open distance F between distal end 44 and base 46 of semi-circular segment 24b of less than or greater than 3.5 centimeters, and the curvature could defines an arc having a less than or greater than 6 centimeter base G extending between distal end 44 and point 48 on semi-circular segment 24b.

In an alternate embodiment, a medium size embodiment may be formed from stainless steel wire of any firmness and/or diameter.

In an alternate embodiment, a medium size embodiment may be formed from any length of wire.

In an alternate embodiment, a small size embodiment may be formed with a radius D of less than or greater than 3.5 centimeters and a diameter E of less than or greater than 7.3 centimeters.

In an alternate embodiment, a small size embodiment may be formed with an open distance F between distal end 44 and base 46 of semi-circular segment 24b of less than or greater than 5.5 centimeters, and the curvature could defines an arc having a less than or greater than 7 centimeter base G extending between distal end 44 and point 48 on semi-circular segment 24b.

In an alternate embodiment, a large size embodiment may be formed from stainless steel wire of any firmness and/or diameter.

In an alternate embodiment, a large size embodiment may be formed from any length of wire.

In an alternate embodiment, a large size embodiment may be formed with a radius D of less than or greater than 4.4 centimeters and a diameter E of less than or greater than 8.9 centimeters.

In an alternate embodiment, a large size embodiment may be formed with an open distance F between distal end 44 and base 46 of semi-circular segment 24b of less than or greater than 6.8 centimeters, and the curvature could defines an arc having a less than or greater than 8.1 centimeter base G extending between distal end 44 and point 48 on semi-circular segment 24b.

In an alternate embodiment, torque handle 60 may be rectangular, square, trapezoidal or any other suitable shape.

In an alternate embodiment, torque handle 60 may be formed without grip-enhancing pattern 64 thereon.

In an alternate embodiment, grip-enhancing pattern 64 of torque handle 60 could be generally horizontally positioned, generally vertically positioned or positioned at any angle relative to handle 60.

In an alternate embodiment, torque handle 60 could carry a stipple pattern, a crosshatch pattern, a plurality of parallel lines, a rubberized coating or any other grip-enhancing means or surface.

In an alternate embodiment, fixation tip 80 could be provided without steroid coating.

In an alternate embodiment, generally arcuately shaped tines 42a and 42b could be positioned wherein first attachment ends 82a and 82b of tines 42a and 42b could not be opposingly positioned.

In an alternate embodiment, tines 42a and 42b could be formed from non-conductive material.

In an alternate embodiment, after deployment tines 42a and 42b could remain generally arcuately shaped.

In an alternate embodiment, core surface 114 within cone body 110 could define a generally cylindrical shape.

In use, ventricular pacing lead device 10 is inserted into a patient through superior vena cava and across the tricuspid valve, preferably with fixation tip 80 in a fully retracted position. Torque handle 60 is gripped to facilitate manipulation of curved lead guide wire or stylet 20, wherein the resiliency of guide wire or stylet 20 provides flexibility of curvature to enable curve loss to assist passage and curve reformation during placement and fixation. Deployment and locking mechanism 100 is activated to enable user control of presentation and fixation of tines 42a and 42b into the heart wall or to the trabeculae of the atrial appendage, wherein tines 42a and 42b are positioned and affixed substantially external to distal end 44 of pacing lead 40.

Compression forces from second deployment spring 118 advance o-ring 126, proximate to outer surface 111 of cone body 110 and threaded member 128, wherein first o-ring locking device 124a and second o-ring locking device 124b retain o-ring 126 therebetween, fixing position of threaded member 128 relative to thread receiver 132 which performs as a deployment mechanism screw cam by generally interacting with threaded member 128 to deploy fixation tip 80 and tines 42a and 42b. Proximal end 106 of pacing lead 40 connects the bipolar component to the pacing generator to complete the insertion and installation process.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. A pacing lead device for ventricular utilization, comprising:
a wire, said wire having a preformed curvature, a proximal end and a distal end;
a handle, said handle positioned proximate to said proximal end of said wire;
a means for fixation of said pacing lead device within the ventricle, said means for fixation carried substantially within a stylet prior to fixation and said means for fixation deployed from within said stylet for fixation.

2. The pacing lead device of claim 1, wherein said means for fixation is a tip positioned proximate to said distal end of said wire, said tip having at least one generally arcuately shaped tine.

3. The pacing lead device of claim 1, wherein said preformed curvature of said wire is an anatomically correct preformed ventricular curve.

4. The pacing lead device of claim 3, wherein the firmness and resiliency of said wire enables curve loss during insertion the ventricle and curve reformation for fixation.

5. The pacing lead device of claim 1, wherein an exterior surface of said handle has grip enhancing features.

6. The pacing lead device of claim 1, wherein said means for fixation is a fixation tip having two generally arcuately shaped barbs carried proximate to distal end of said pacing lead device.

7. The pacing lead device of claim 6, wherein said two generally arcuately shaped barbs are opposingly positioned.

8. The pacing lead device of claim 3, wherein said anatomically correct preformed ventricular curve is defined by a first generally linear segment and a second generally semi-circular segment.

9. The pacing lead device of claim 7, wherein said fixation tip has a steroid coating.

10. The pacing lead device of claim 2, wherein said stylet has a hollow longitudinal core and a conductive connector is carried therein, proximate to said proximal end of said wire, wherein said tip has a conductive, conically shaped body having a receiving port, said receiving port for receiving said proximal end of said wire, and wherein a threaded member is carried proximate to said receiving port of said tip.

11. The pacing lead device of claim 10, wherein said hollow longitudinal core has a surface, said surface having a coating.

12. The pacing lead device of claim 10, further comprising a retraction spring, a first o-ring lock, a deployment spring, a second o-ring lock, a thread receiver and an o-ring, said o-ring carried proximate to said threaded member.

13. The pacing lead device of claim 12, further comprising an electrical conducting strip carried proximate to said receiving port of said conically shaped body, a first wire coil conductor, said first wire coil conductor carried proximate to said hollow longitudinal core, and a second wire coil conductor, said second wire coil conductor substantially encircling said first wire coil conductor.

14. The pacing lead device of claim 13, wherein said first wire coil conductor has an insulating coating.

15. The pacing lead device of claim 14, further comprising a bipolar electrical band carried proximate to second wire coil conductor.

16. The pacing lead device of claim 15, further comprising a suture anchor sleeve carried on an outer surface of said pacing lead device proximate to said proximal end.

17. A semi-circularly shaped ventricular pacing lead device comprising:
a substantially resilient stylet wire having a proximal end and a distal end;
a torque grip, said torque grip carried proximate to said distal end of said stylet wire and said torque grip having a grip enhancing surface;
an active fixation tip, said active fixation tip carried proximate to a proximal end of said stylet wire and said active fixation tip having a substantially conical shape with two semi-helical tines;
a deployment and locking mechanism for said active fixation tip, said deployment and locking mechanism carried substantially within said stylet wire,
wherein a conductive connector is carried substantially within a core of said stylet wire, said core having a coating and said core extending into said active fixation tip, wherein a first conductive spring is carried proximate to said core and a second conductive spring generally surrounds said first conductive spring, wherein a bipolar electrical band is carried proximate to said second conductive spring, and wherein a threaded member, a retraction spring, a deployment spring, a screw cam, an o-ring, and at least one o-ring lock are carried proximate to said active fixation tip and cooperate to activate said fixation tip.

18. The method of insertion and fixation of a ventricular pacing lead for utilization with a pacing generator, comprising the steps of:
a. obtain a ventricular pacing lead device having a preformed curvature, a torque handle and a deployable active fixation tip having semi-helical tines;
b. retract said deployable active fixation tip;
c. grip said torque handle to facilitate manipulation and passage of said deployable active fixation tip into the ventricle;
d. deploy said active fixation tip;
e. affix said semi-helical tines to the heart wall or to the trabeculae of the atrial appendage;
f. conductively connect said ventricular pacing lead device to the pacing generator.
